(19) United States
(12) Patent Application Publication
(10) Pub. No.: US 2021/0059870 A1
(43) Pub. Date: Mar. 4, 2021

(54) DIAPER WITH ANATOMICAL SEPARATOR AND POCKET

(71) Applicant: Lissette Wezley Yllanes Nauca, Huanuco (PE)

(72) Inventor: Lissette Wezley Yllanes Nauca, Huanuco (PE)

(21) Appl. No.: 16/960,068

(22) PCT Filed: Feb. 21, 2018
(86) PCT No.: PCT/PE2018/000005

§ 371 Date: Jul. 3, 2020

(30) Foreign Application Priority Data
Jan. 3, 2018 (PE) 0001-2018/DIN

(51) Int. Cl. A61F 13/495; B32B 5/02; B32B 5/24; B32B 27/12; B32B 27/32; A61F 13/84; A61F 13/56; A61F 13/514

(52) U.S. Cl. CPC A61F 13/495; B32B 5/022; B32B 5/24; B32B 27/12; B32B 27/32; A61F 13/84; A61F 2013/4951; A61F 13/514; B32B 2555/02; B32B 2307/726; B32B 2262/02; B32B 2307/7265; B32B 2262/062; A61F 13/5633

(57) ABSTRACT

A double-layer diaper with anatomical separator and pocket comprising: an outer cover, an absorbent layer, an upper layer, an anatomical separator cover, an anatomical separator, a few closure side panels, and a pocket; wherein the core portion of invention is the anatomical separator, which prevents stool and urine deposited in a diaper from mixing and causing several diseases in infants, such as rash, urinary tract infections, and prevents microorganism generation; additionally, the pocket may be used to store baby wipes facilitating its use and cleaning of baby genitalia.

### TECHNICAL FIELD

The present invention falls within the technical field of absorbent pads, specifically a diaper-like absorbent clothing used to sanitize and prevent environment pollution due to body wastes.

### STATE-OF-THE-ART (OR BACKGROUND OF THE INVENTION)

Currently, there are several disposable adsorbent clothes that serve the purpose of a diaper, which is to contain and keep the one using it dry and comfortable for several hours; when the diaper is used and it reaches its maximum capacity, it is disposed and replaced with a new one.

Improvements developed for diaper-like absorbent items have been related to characteristics such as the improved securement of diapers onto the body of the baby, anatomical aspects, improved leg stretching and waist fitting means, shape of materials that regulate, lead and control liquid flow in diaper, improved adhesive securing tapes of diaper, use of gels or other products to encapsulate urine and increase liquid retention capacity, among others.

In the state-of-the-art, AR092705A1 (“DISPOSABLE DIAPER”) patent document was verified. It describes a disposable diaper including a pair of leg stretching units that spread on the leg opening, which may expand and contract in longitudinal direction in the product; the separation distance between the end of the leg stretching units in the rearward waist line area 30 is longer than the separation distance between the end of the leg stretching units in the forward waist line area; the leg stretching units that spread from the crotch area to the rearward waist line area are convex towards the exterior in the direction of the product width; the leg stretching units that spread from the crotch area to the forward waist line area are convex towards an interior face in the direction of the product width.

In addition, ES2571227T3 (“Reusable diapers”) patent document, which describes a gender neutral reusable diaper comprising: an inner layer comprising an absorbent material; an outer layer formed by a significantly impermeable material; at least one fluid-absorbing insert for use in absorbing fluids; one forward waist portion defined by at least part of the inner layer; one rearward waist portion defined by at least part of the inner layer; and one crotch portion disposed generally between the forward waist portion and rearward waist portion; wherein the inner layer is disposed generally between the outer layer and the at least one fluid-absorbing insert; wherein the at least one fluid-absorbing insert has a first end and a second end opposite the first end, the first end is secured to the inner layer at a first location adjacent to the forward waist portion and the second end is secured to the inner layer at a second location adjacent to the rearward waist portion; wherein the at least one fluid-absorbing insert includes two individual and separate layers having free ends that are overlapped and stitched to the inner layer; and wherein the at least one fluid-absorbing insert includes a portion between the first and second end that is adjustable relative to the inner layer between the first location and the second location, for selectively changing an overlapped region of the at least one fluid-absorbing insert to adapt to the use by a male and/or a female, where the layers of the at least one fluid-absorbing insert are folded, bulked or united over themselves to provide the overlapped region.

WO9728774A1 (“DIAPERS HAVING PRECOMBINED MECHANICAL CLOSURES”) patent document was also verified. It discloses a diaper that has a multilayer tab or tape assembly arranged to form the union of the user with a mechanical securement portion and to provide at least one adhesive or adhesive securement portion to maintain tab stability as the tab is assembled with the diaper and during tab storage on the diaper prior to tab deployment. The adhesion securement portion includes a separation interface or separable bond portion that is separable to provide non-adhesive exposed surfaces on tab deployment to the securement position; the separable interface or separable portion has a transferable release coat that remains with and covers an otherwise exposed adhesive surface, a non-adhesive sensitive adhesive or cohesive, or separable or peeling films; a central finger lifting may be provided to facilitate proper layer separation and deployment of the tab during use.

The MX2009008544 background was also identified, which refers to a disposable diaper comprising a separator sheet for passage of bodily wastes, in a front region thereof with a front opening for passage of bodily wastes and in a rear region thereof with a rear opening for passage of bodily wastes, where said separator sheet includes a middle region defined between said front opening and said rear opening and adapted to separate itself upwards to prevent the inner absorbent sheet from getting in contact with the diaper user skin. In said background document, it is stated that the main invention objective is to prevent contamination with excrement of a skin portion between external genitalia and the anus of a disposable diaper user. However, said separator sheet does not appear to be capable of creating an angular space between separator sheet and diaper for retention of excrements and urine, in such a way that it is sufficiently suitable to prevent a large volume of excrement from going to the other side of the separator sheet, and said separator sheet does not appear to have an absorbent cover either to prevent humidity from transferring to said separator sheet.

Thus, the above background products do not have a proper separation of stool from urine; said disadvantage results in diseases caused by the above-mentioned diapers, including:
Rash: Eruptions produced when the baby skin gets in contact with urine and stool normally during long periods, humidity is the most common cause behind the diaper, because when diapers are dirty, they do not fully absorb excreted residual liquids.
Generation of Microorganisms (bacteria and fungi): Infections due to fungi such as Candida albicans are seen in 80% of neonates with skin irritation around the diaper area; a wet diaper leads to a rupture of skin barriers, and infection usually occurs within 48-72 hours after irritation.
Urinary Tract Infection (UTI): It is a common problem in infants and toddlers. The main causal agent of UTI, in both girls and boys, is E. coli. The infection route is almost always upwards, from microorganisms coming from the intestine that are located in the perineal area going upwards through the urethra to the bladder.

For all of the above, a diaper is needed with the capacity to properly separate stool from urine, including a large amount of stool, in order to look after the health of babies and prevent genitourinary infections, such as those already mentioned.

### DESCRIPTION OF THE INVENTION

This invention was developed as a solution for the above-mentioned problems, and it consists of a diaper with anatomical separator that in a preferred modality may include a pocket and a double filter layer, which main objective is to prevent genitourinary infections, rash and dermatitis caused by state-of-the-art diapers, since the proposed diaper comprises the following portions:
At least one outer cover: It is preferably made of a thermoplastic-type material, made of polyolefin or in other preferred modality of an impermeable material. It is in charge of housing all other portions contained in the diaper, as follows:
   a. The upper side of the outer cover houses the absorbent layer, anatomical separator cover and anatomical separator, and it may optionally include the upper layer.
   b. Optionally, the pocket is in the cover rear side, which is also made of an impermeable material to prevent potential leaks when containing urine and stool.
At least one absorbent layer: It is preferably made of a polypropylene, cellulose type material and a superabsorbent polymer; cellulose quickly absorbs and transfers urine to the superabsorbent polymer, where it is trapped. It is located on the outer cover; this layer is in charge of fluid storage.
At least one upper layer: This element is optional and refers to an absorbent film lined with soft fibers, similar to clothing, which helps preventing humidity from transferring to the baby; it is located between the absorbent layer and anatomical separator. This layer is in charge of transferring fluids quickly to the absorbent layer while it remains soft and dry to the touch.
At least one anatomical separator cover: It is an absorbent film lined with soft fibers, similar to clothing, which helps preventing humidity from transferring to the anatomical separator. This element covers the anatomical separator.
At least one anatomical separator: It is bulky shaped and arranged lengthwise from side to side of the crotch sides in the central portion of the diaper, preventing stool from moving into the genital area. The separator is preferably cylindrical in shape, made of a polyolefin type material filled with silicone fleece to provide thickness, prevent it from moving with body movement and be able to retain stool properly, thus allowing no passage of stool to the genital area. The bulk cylindrical shape allows creating an angular space for retention of stool in one side and urine in the other.
At least a pair of closure side panels: They are adhered to the sides of the upper end of outer cover to provide a mechanical gripping and are preferably made of a polypropylene adhesive tape type material, which are hooked into the lower ends of the outer cover by adhesive or other means.
At least one pocket: This element is optional and is located on the outer cover rear portion; the pocket may house a series of wipes or other elements that will help improve cleaning of infants, rationalize the exact amount of wipes by not carrying the whole wipe package in the diaper bag, and making it more comfortable and practical, because it is of a polyolefin type material, and thus, it allows preserving wipes wet.

This involves an improvement in the state-of-the-art, as the present invention does solve the problem of separation between stool and urine by an anatomical separator like a physical barrier integrated in the diaper, which prevents stool from sliding to the baby genitalia; the anatomical separator preferably has a cylindrical shape that creates an angular space for a more efficient retention of stool, thus reducing genitourinary infections; additionally, the outer layer contains at least one pocket that might house wet wipes to complete baby cleaning.

### BRIEF DESCRIPTION OF THE FIGURES

The following figures are for illustrative purposes only to better understand the present invention and are not intended to limit the scope of claims in any way.

FIG. 1: An isometric view of the double-layer diaper with anatomical separator and a pocket is shown.

FIG. 2: A side view of the double-layer diaper with anatomical separator and pocket is shown.

FIG. 3: A blown-up view of the double-layer diaper with anatomical separator and pocket is shown.

FIG. 4: An isometric lower view of the double-layer diaper with anatomical separator and pocket is shown.

FIG. 5: An upper view of the double-layer diaper with anatomical separator and pocket is shown.

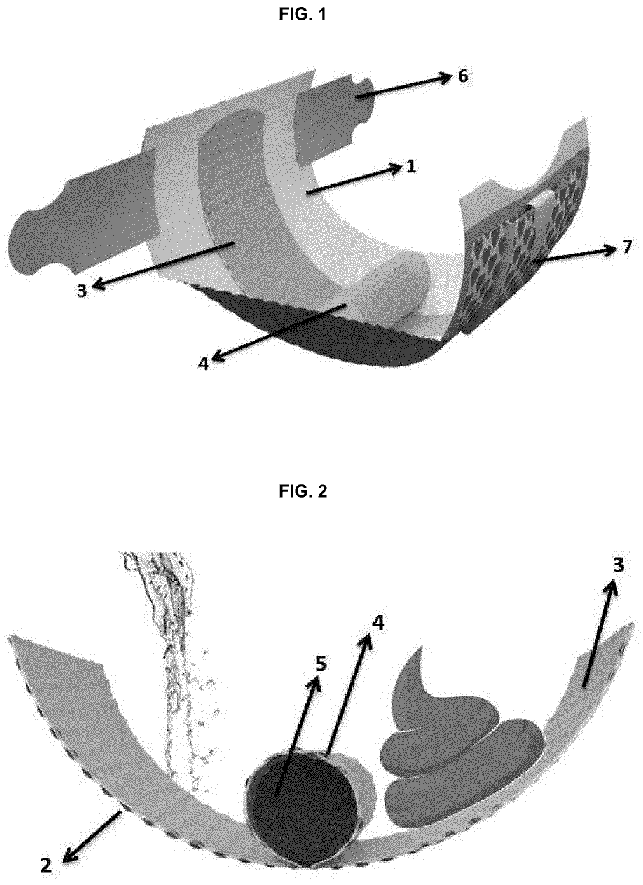

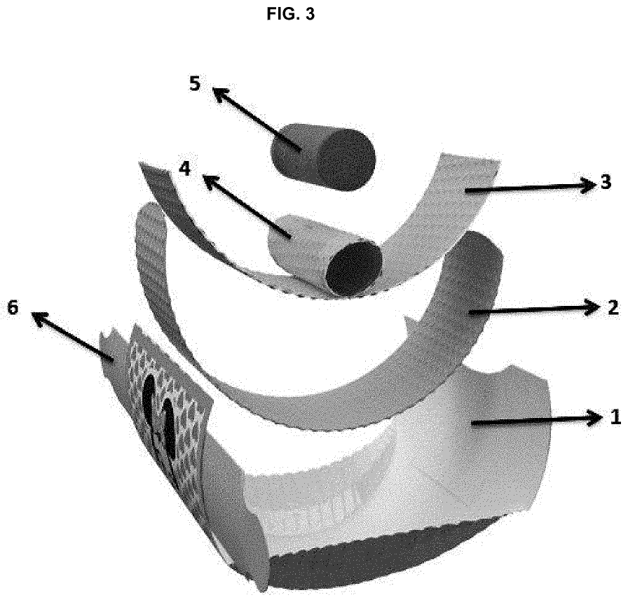

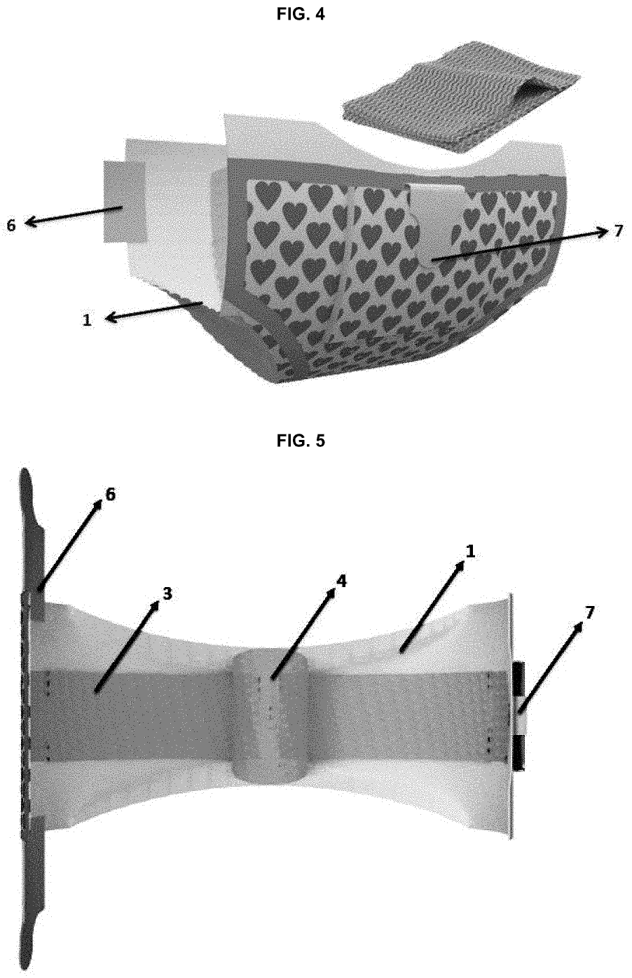

### PARTS OF THE FIGURES (CAPTION)

1. Outer cover

2. Absorbent layer

3. Upper layer

4. Anatomical separator cover

5. Anatomical separator

6. Stretching and closure side panels

7. Pocket

### PREFERRED EMBODIMENT OF THE INVENTION

According to the above, the present invention in a preferred form is a double-layer diaper with anatomical separator and pocket comprising: an outer cover (1), an absorbent layer (2), an upper layer (3), an anatomical separator cover (4), an anatomical separator (5), a few closure side panels (6), and a pocket (7); wherein the core portion of invention is the anatomical separator (5), which prevents stool and urine deposited in a diaper from mixing and causing several diseases in infants, such as rash, urinary tract infections, and prevents microorganism generation.

It may also be designed as adult diapers, specifically for those persons who do not have or have lost the ability to control their waste (stool and urine), or are unable to find a place to deposit it. The industrial application it may be given is for people who cannot reach a bathroom on time or a bathroom is not accessible, such is the case of astronauts, divers, vehicle drivers, etc.

In other preferred embodiments, diapers may include more or less than two layers or films with absorption capacity.

What is claimed is:

1. A type of diaper comprising at least one outer cover (1), at least one absorbent layer (2) and at least one pair of closure side panels (6), characterized by the fact that the outer cover that is responsible for housing: the absorbent layer (2), at least one anatomical separator (5), and at least one anatomical separator cover (4); wherein the anatomical separator cover (4) is an absorbent film that covers the anatomical separator (5) and helps to prevent humidity from being transferred to the anatomical separator (5); additionally, wherein said anatomical separator (5) takes a cylinder shape and is arranged lengthwise from side to side of the crotch sides in the central portion of diaper, preventing stool from moving into the genital area.

2. (canceled)

3. The diaper, according to claim 1, characterized by the fact that the anatomical separator is made of polyolefin-type material, filled with silicone fleece.

4. The diaper, according to claim 1, characterized by the fact that the rear side of outer cover (1), houses at least one pocket (7) to contain wipes or other elements that will help to improve cleanliness of the infant.

5. The diaper, according to claim 1, characterized by the fact that the outer cover is of a thermoplastic-like material, made of polyolefin.

6. The diaper, according to claim 1, characterized by the fact that the outer cover is made of an impermeable material.

7. The diaper, according to claim 1, characterized by the fact that between absorbent layer and anatomical separator there is an upper layer (4), which is an absorbent film lined with soft fibers.

8. The diaper, according to claim 1, characterized by the fact that the absorbent layer (2) is made of polypropylene, cellulose and a super-absorbent polymer, this layer is responsible for storing fluids and is located on the upper side of the outer cover (1).

9. The diaper according to claim 1, characterized by the fact that the closure side panels (6) are adhered to the sides of the upper end of the outer cover (1) to provide a mechanical grip, and they are polypropylene adhesive tapes, which are hooked into the lower ends of the outer cover (1) by adhesive or other means.
